(19) United States
(12) Patent Application Publication
(10) Pub. No.: US 2016/0376595 A1
(43) Pub. Date: Dec. 29, 2016

(54) PEPTIDE APTAMERS FOR MANIPULATING PROTEIN FUNCTION

(71) Applicant: Purdue Research Foundation, West Lafayette, IN (US)

(72) Inventors: Stanton B. Gelvin, West Lafayette, IN (US); Lan-Ying Lee, West Lafayette, IN (US)

(73) Assignee: Purdue Research Foundation, West Lafayette, IN (US)

(21) Appl. No.: 15/260,461

(22) Filed: Sep. 9, 2016

Related U.S. Application Data
(62) Division of application No. 13/823,895, filed on Mar. 15, 2013, now abandoned.
(60) Provisional application No. 61/432,944, filed on Jan. 14, 2011.

(51) Int. Cl. C12N 15/115; G01N 33/53; C12N 15/82

(52) U.S. Cl. CPC C12N 15/115; C12N 15/8218; G01N 33/5308; C12N 2310/16

(57) ABSTRACT

Peptide aptamers and the methods to produce cassettes including the aptamers and manipulating them, are described. The peptide aptamer cassettes are useful to, e.g., inhibit protein function such as proteins necessary for the transformation of plants, or to replicate cells.

### CROSS REFERENCE TO RELATED APPLICATIONS

This patent application claims priority from copending U.S. Provisional Application 61/432,944 filed Jan. 14, 2011, the content of which is herein incorporated by reference in its entirety.

The United States Government has rights in this invention pursuant to National Science Foundation (NSF) Grant Contract No. 103735 and CBPR Grant No. GO12026-318, between the United States Government and Purdue Research Foundation.

### SEQUENCE LISTING

The instant application contains a Sequence Listing which has been submitted in ASCII format via EFS-Web and is hereby incorporated by reference in its entirety. Said ASCII copy, created on Jan, 10, 2012, is named 116290_SEQ_ST25.txt and is 25,144 bytes in size.

### BACKGROUND

Aptamers are nucleic or amino acid macromolecules that may he designed to bind tightly to specific targets. Targets include structures from proteins to small organic dyes. In solution, a chain of nucleotides forms intramolecular interactions that fold the molecule into a complex three-dimensional shape that allows it to bind tightly against the surface of its target molecule.

Peptide aptamers are short peptides of random amino acid sequences. As commonly used, these peptides are generally 15-20 amino acids-long. This length provides enough flexibility for the peptide to assume various conformations, while reducing the probability of randomly creating a stop codon in the aptamer coding sequence.

Aptamers can be “free” (i.e., as a “tag” on the end of another protein) or “constrained” (i.e., inserted between two other polypeptides). Constraining aptamers apparently lowers their free energy of folding and allows them more easily to take on conformations conducive to interactions with other proteins.

Peptide aptamers have a number of biological uses: as “tags” to identify interacting proteins (e.g., using Bi FC, bimolecular fluorescence complementation), as “mutagens” to disrupt functionality of proteins by binding and thereby inhibiting activity (e.g., acting as competitive inhibitors for substrate or peptide binding), as a way to identify specific domains of proteins by binding to and inhibiting certain protein functions without necessarily inhibiting all functions of the target protein, and as a bioinformatics tool to identify interacting partners of target proteins (e.g., the sequence or structure of an interacting peptide aptamer may give clues as to what proteins may also interact with the target protein).

### SUMMARY

A peptide aptamer technology to investigate and manipulate protein function in vivo was developed. Aptamer expression “cassettes” were developed in which aptamers are constrained between components of autoflourescent proteins that allow detection of target proteins and may affect their function. An advantage of the methods and compositions disclosed herein is not only to determine when aptamers enter cells, but also when they interact with target proteins. Strong promoters (for example in plants, the CaMV double 35S promoter, in animals, the CMV promoter) may drive expression of the novel “polyprotein” cassettes containing the aptamers. A suitable polyprotein includes an autofluorescent protein-aptamer (e.g. a 20 amino acid peptide)-complementary autofluorescent protein fragment. A polyA addition signal may follow. A suitable autofluorescent protein is mCherry, a suitable complementary autofluorescent protein fragment is nVenus. Alternatively, a suitable polyprotein is cYFP-aptamer-mCherry. In this situation, the interaction of the aptamer can be detected using BiFC, with a protein tagged with nYFP or nVenus. Expression of the aptamer in cells results in fluorescence (e.g. red from mCherry).

The system can be set up for bimolecular fluorescence complementation (BiFC) if a known target protein is tagged with cCFP or cYFP (if nYFP or nVenus is part of the polyprotein), or if a known target protein is tagged with nYFP or nVenus (if cYFP or cCFP is part of the polyprotein). Interaction of the aptamer with the target protein can generate yellow fluorescence resulting from correct folding of nVenus/nYFP and cCFP/cYFP. A series of vectors contain restriction endonuclease sites between mCherry and nVenus, into which the aptamer can be cloned. The system is adopted for Gateway® Recombination Cloning Technology use, or a suitable cloning method, achieving the same goals, as known to those of skill in the art.

Separately, a Gateway®-compatible library of 2×10⁸ random aptamer sequences was generated for incorporation into the final aptamer expression vectors.

As proof of concept, >10 aptamers were generated that were directed against the Agrobacterium virulence effector protein, VirE2. When introduced into plant cells with VirE2-cCFP, many aptamers generate yellow fluorescence, indicating aptamer interaction with the VirE2 target protein. Hundreds of transgenic Arabidopsis plants were generated expressing various aptamers targeted against VirE2. When roots of these transgenic plants were challenged with infection by Agrobacterium, plants containing some (but not all) aptamers were more resistant to infection than were wild-type plants.

Uses of peptide aptamers includes generation of a phenotype (aptamer “mutagenesis”) without mutating the genome. This is done by aptamers interacting with target proteins and inhibiting protein function. Thus, they are used to inhibit any protein or function for which there is a detection assay, providing wide applications. Aptamers in cassettes as disclosed herein can inhibit Agrobacterium-mediated plant transformation. They may inhibit aggressive mobility of cancer cells, targeting suitable proteins. Expression of toxic compounds may be inhibited. The system is not limited to plants.

The aptamer expression system disclosed herein is unique. There are no reports of autofluorescence and BiFC used with aptamer or Gateway® technology. Thus, the system is uniquely set up to maximize ease of use and multiple applications.

### BRIEF DESCRIPTION OF THE DRAWINGS

FIG. 1: Features of an embodiment of a peptide aptamer expression cassette. 2X35S represents the Cauliflower Mosaic Virus double 35S promoter; TL, the tobacco etch virus translational leader; mcs, the multiple cloning site; ter, the Cauliflower Mosaic Virus 355 polyA addition signal.

FIG. 2: Bimolecular Fluorescence Complementation (BiFC) (Hu et al., 2002).

FIG. 3: Detection system for aptamer-protein interactions in plants using multi-color bimolecular fluorescence complementation (BiFC).

FIG. 4: Aptamer expression “cassettes” can be loaded into a common plasmid for interaction studies in cells (see FIG. 1 for symbols).

FIG. 5: Mutagenesis of octopine-type VirE2 protein. The black vertical lines are the nuclear localization signal (NLS) sequences.

FIG. 6: Domains of VirE2 from which aptamers were generated. The black vertical bars are the positions of the aptamers.

FIG. 7: The “empty” aptamer polyprotein localizes to the nucleus and cytoplasm of transfected BY-2 protoplasts. The 4 images are of the same cell clusters under (A) brightfield, or (B, C, D) fluorescence microscopy to image mCherry (RFP, red, fluorescence protein) or DAFT (4′, 6-diamidino-2-phenylindole), which stains DNA in the nucleus. D=RFP and DAPI overlay.

FIG. 8: The mid-80 aptamer localizes to the nucleus and cytoplasm, but interacts with VirE2 only in the cytoplasm. Upper and lower photos are of two different cells.

FIG. 9: Sites of interaction of VirE2 with various aptamers (A) VirE2 multimerizes in the cytoplasm aptamer expression cassette, (B) Aptamer 5 interacts with VirE2 in the cytoplasm, but also localizes to the nucleus, (C) Aptamer 6 strongly interacts with VirE2 in the cytoplasm, and remains there.

FIG. 10: Aptamer expression cassettes can be loaded into a T-DNA binary vector and expressed in transgenic plants. Plants expressing these aptamers were infected with Agrobaterium to determine if aptamer expression would inhibit transformation: Abbreviations are as in FIG. 1 legend, plus RB, T-DNA right border; LB, T-DNA left border.

FIG. 11: Expression of aptamers 2, 5, 6, & 7 inhibits transformation of T1 plants. Dark bars are results of individual sets of experiments; shaded bars are the average of these experiments for each aptamer. Numbers above the bars indicate the number of individual transgenic plants examined for each aptamer.

FIG. 12: T2 generation data confirm transformation inhibition by aptamers.

FIG. 13: Transformation susceptibility of T2 generation aptamer lines. These are pictures of root segments from transgenic lines expressing various aptamers; e.g., 6-7 means aptamer 6, line number 7. The root segments have been infected with the oncogenic strain A. tumefaciens A348 at 10⁷ cfu/ml, and the development of tumors was recorded one month later. Many lines are resistant to transformation, whereas some are still susceptible.

FIG. 14: Location of aptarners on the VirE2 amino acid sequence (Dym et al., 2008.) FIG. 14 discloses SEQ ID NOS: 1 and 2, respectively, in order of appearance.

FIG. 15: Plant random aptamer library expression system. Gateway® attL-flanked oligomers encoding random 20 amino acid aptamers are inserted as part of the polyprotein.

FIG. 16: pSAT1-mCherry-nVenus (pE3370).

FIG. 17: Double stranded nucleic acid sequence of FIG. 16. FIG. 17 discloses SEQ ID NOS: 3-5, respectively, in order of appearance.

### DETAILED DESCRIPTION

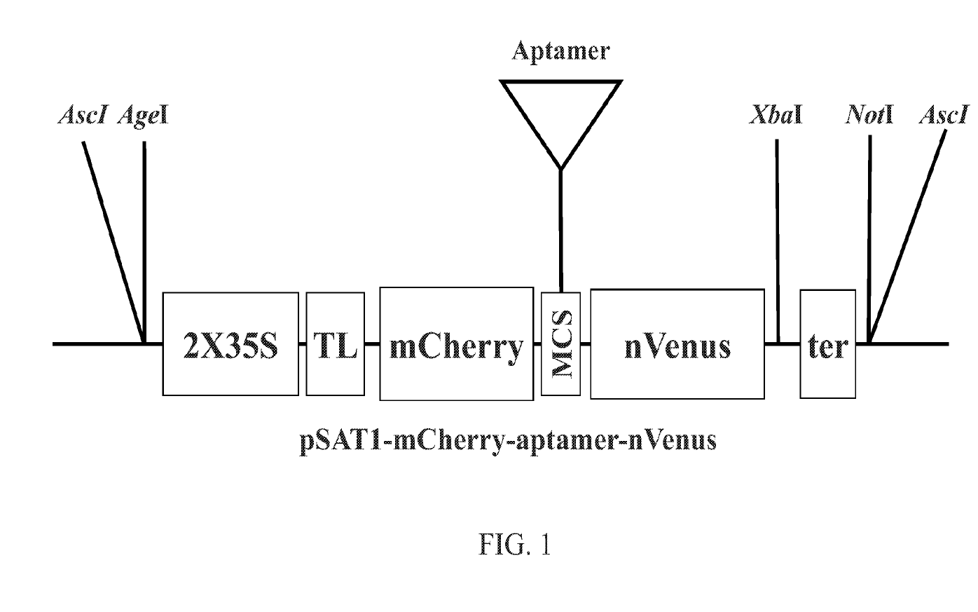

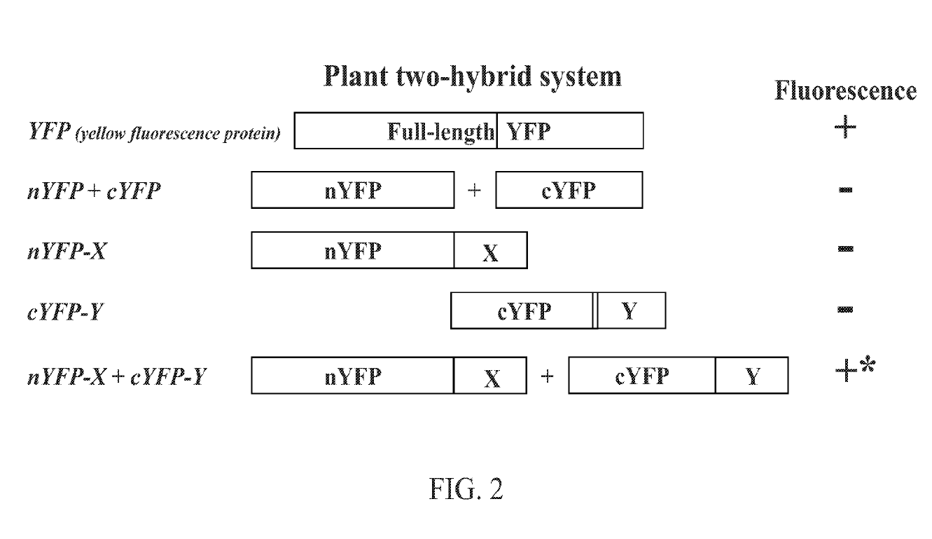

An “expression cassette” was developed to express peptide aptarners in cells. This cassette constrains aptamers between proteins, e.g., an autofluorescent protein, such as mCherry (GenBank Accession Number AY678264) which will generate red fluorescence in transformed cells, and a complementing autofluorescent protein fragment, e.g. nVenus (FIG. 1). If the aptamer interacts with a protein tagged with cCFP, this generates yellow fluorescence (FIG. 2). In this approach, a molecule of yellow spectral variant GFP (YFP) is separated into two portions, N-terminal (nYFP) and C-terminal (cYFP) neither of which fluorescence when expressed alone. Fluorescence is restored when nYFP and cYFP refold, as they are brought together as fusions with interacting (target) proteins. The methods and compositions of the present disclosure not only use aptamers to detect, but also to affect protein function (expression).

Ten different 20-mer aptamers (and one 80-mer) that target potential protein interaction sites of VirE2 were inserted into cassettes.

An aptamer polyprotein interacts with VirE2 in transiently transfected tobacco BY-2 protoplasts and generates yellow fluorescence in the cytoplasm. A control polyprotein (“empty aptamer vector”) does not interact with VirE2.

Several hundred transgenic Arabidopsis plants that express the aptamer polyproteins were generated. Assays of T1 and T2 generation plants for transformation susceptibility indicate that expression of aptamers designated 2, 5, 6, and 7 inhibit transformation. Aptamer 6 is especially inhibitory.

FIG. 1 shows the aptamer is “constrained” as a translational fusion between two peptides. mCherry confers red fluorescence upon cells containing the aptamer construct. nVenus may interact with a cCFP tag on the target protein to generate yellow fluorescence. The entire expression cassette is flanked by Ascl sites for insertion into various vectors.

BiFC approach is based on complementation between two nonfluorscent fragments of the yellow fluorescent protein (YFP) when they are brought together by interactions between proteins fused to each fragment. FIG. 2 shows reconstitution of fluorescence takes place only when the two fragments of the split fluorophore are brought together by protein-protein interaction. The split point of YFP can be at either of the following positions:

nYFP: 1-174 a.a. or nYFP: 1-154 a.a.

cYFP: 175-239 a.a. cYFP: 155-239 a.a.

For these experiments, nVenus₁₋₁₇₄ and cCFP₁₅₅₋₂₃₉ were used.

As proof of concept, VirE2 was chosen as a target protein because: VirE2 is important for Agrobacterium-mediated transformation of plants, and defining its functions provides useful information on the mechanisms of transformation. VirE2 is known to interact with both plant and Agrobacterium proteins in vivo, including other VirE2 molecules, VirE1, VirD4, VIP1, VIP2, and various importin a proteins. In addition, VirE2 presumably interacts with T-strands in plants. Protein interacting domains of VirE2 have been mapped in yeast. A crystal structure of VirE2 in a complex with VirE1 (Dym et al., 2008) serves as a guide for VirE2-VirE2 and VirE2-DNA interactions.

Suitable target proteins are those for which a detection assay is available.

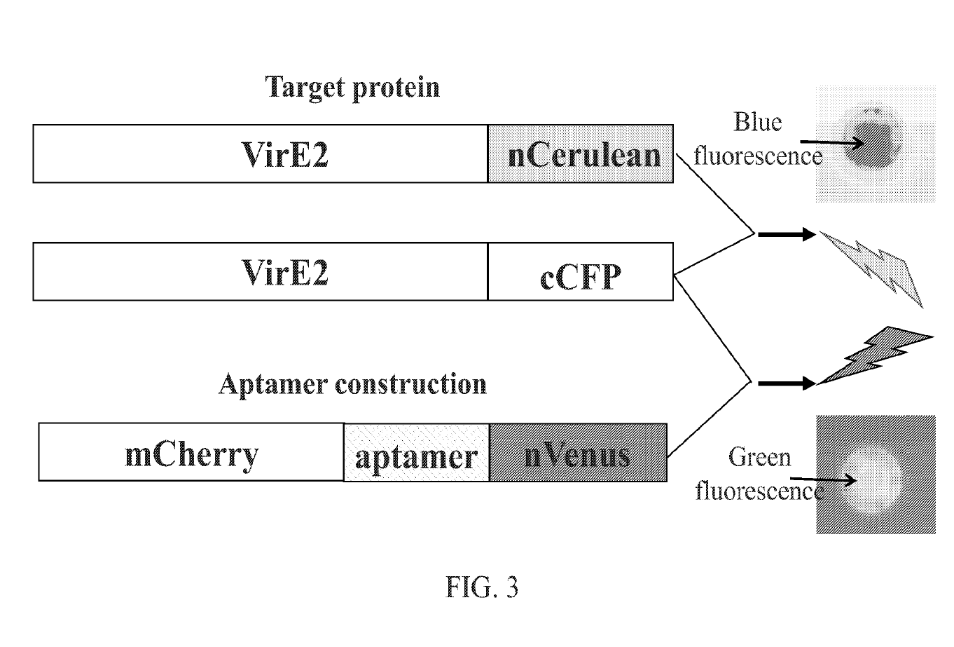

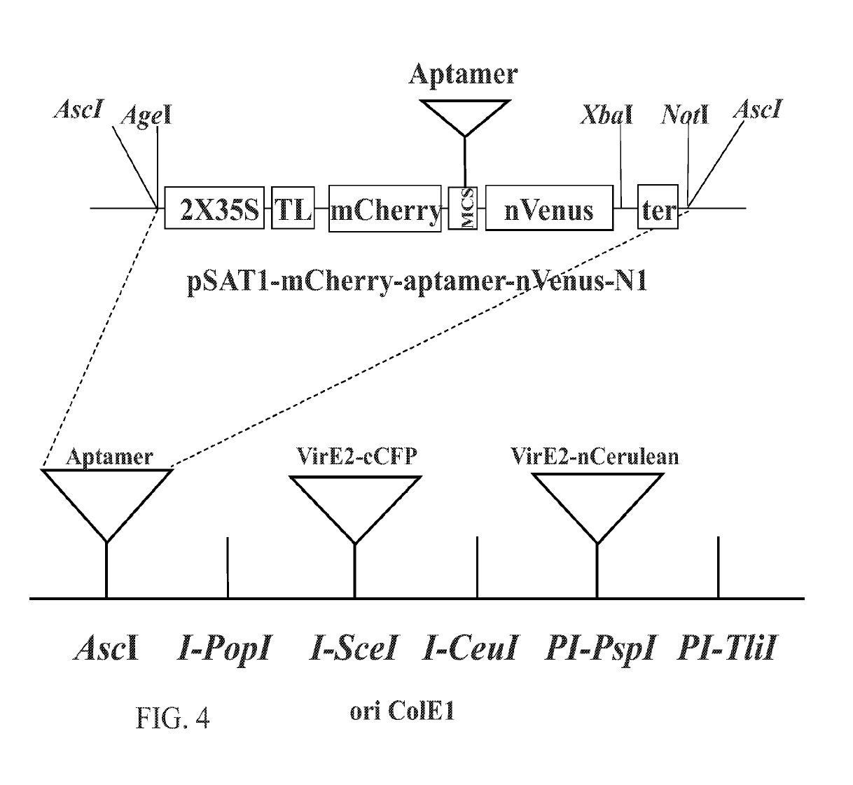

In FIG. 3 the presence of the aptarner in the entire nucleus and cytoplasm of the cell will generate the red fluorescence from mCherry (not shown). If the aptamer interacts with VirE2, green or yellow fluorescence will appear (shaded arrow). VirE2-VirE2 interactions will fluoresce blue (clear arrow). Because of competition of the aptamer with VirE2-VirE2 interactions, blue fluorescence may also decrease. Alternatively, VirE2 self-interaction may diminish aptamer-VirE2 interaction.

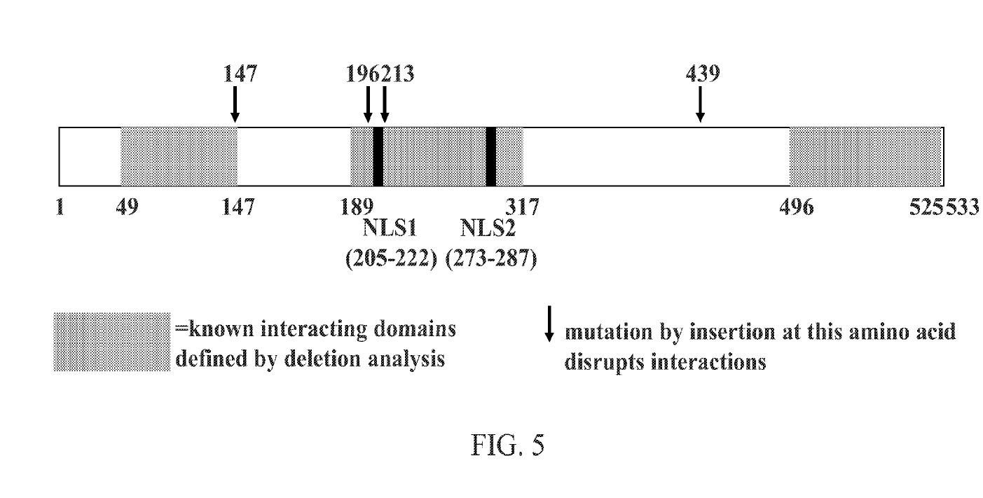

FIG. 5 shows that VirE2 protein domains responsible for VirE2-VirE2 and VirE2-VirE1 interactions have been mapped, mostly using yeast 2-hybrid systems.

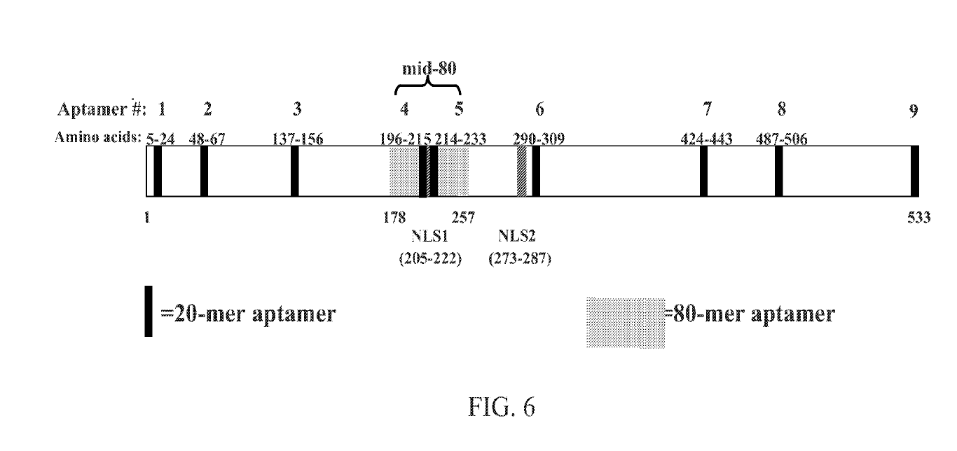

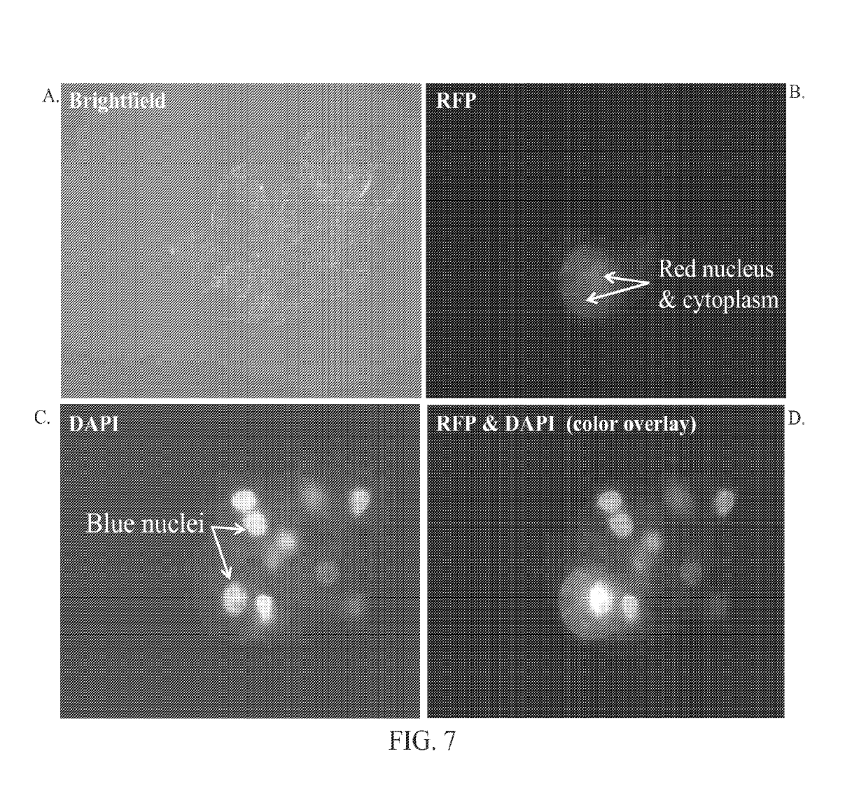

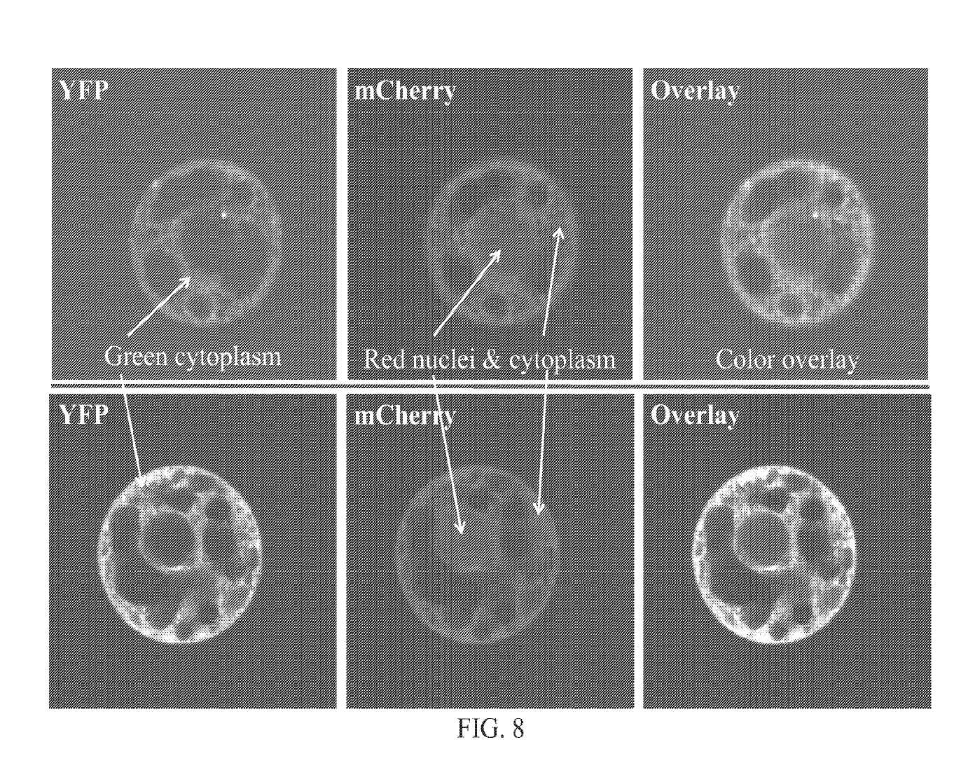

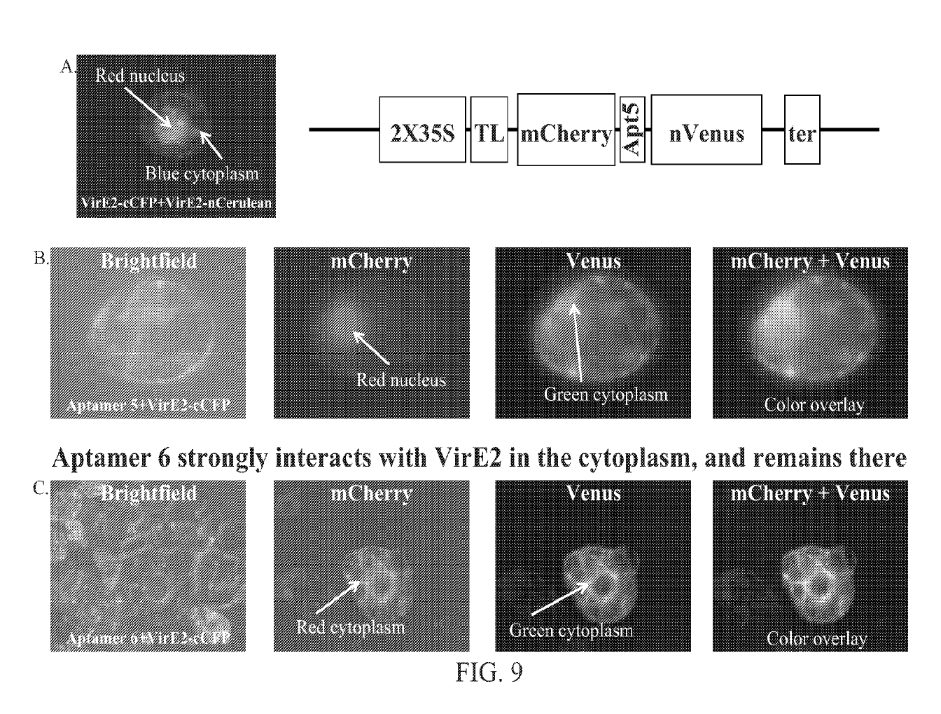

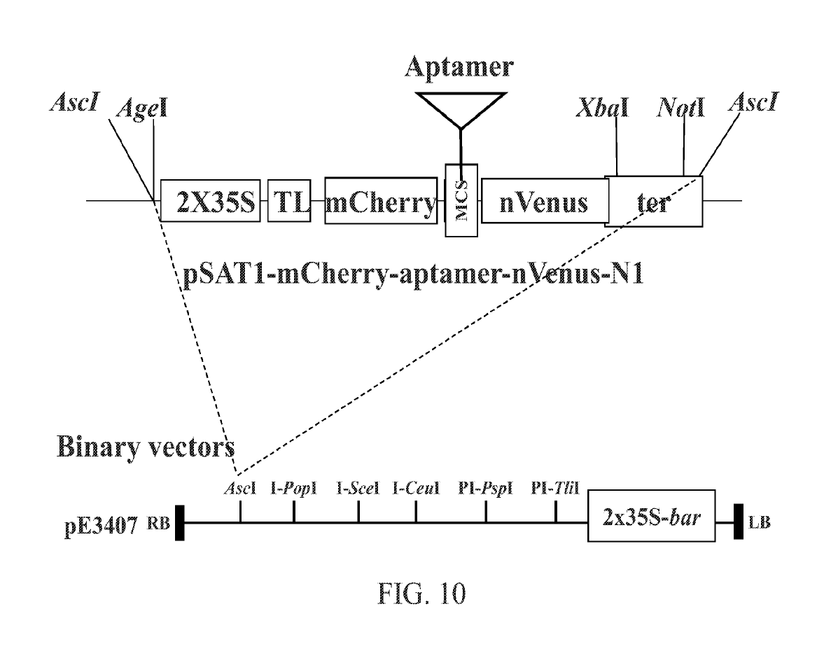

FIG. 6 shows the sites of VirE2 chosen to generate 20 amino acid aptamers. Domain 1 is not conserved among VirE2 proteins. Domains 2-8 represent regions important for VirE2 interactions. Domain 9 contains the T4SS secretion signal. The mid-80 aptamer (a 80 amino acid aptamcr) domain covers one NLS and important interacting domains.

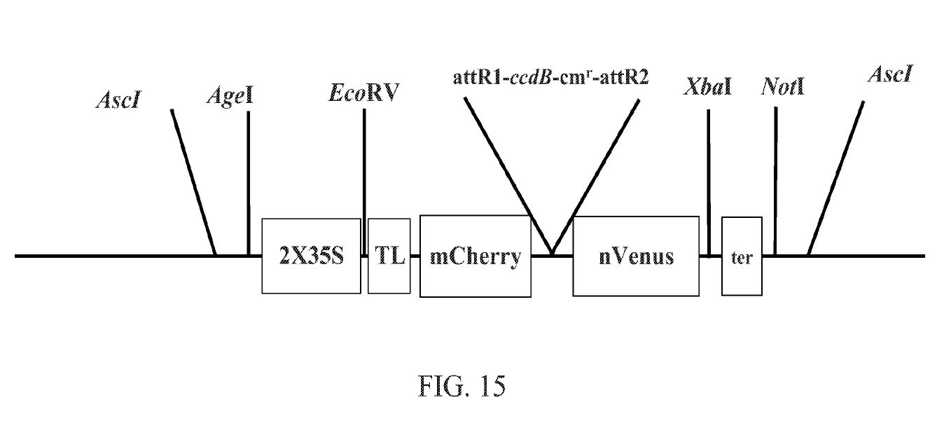

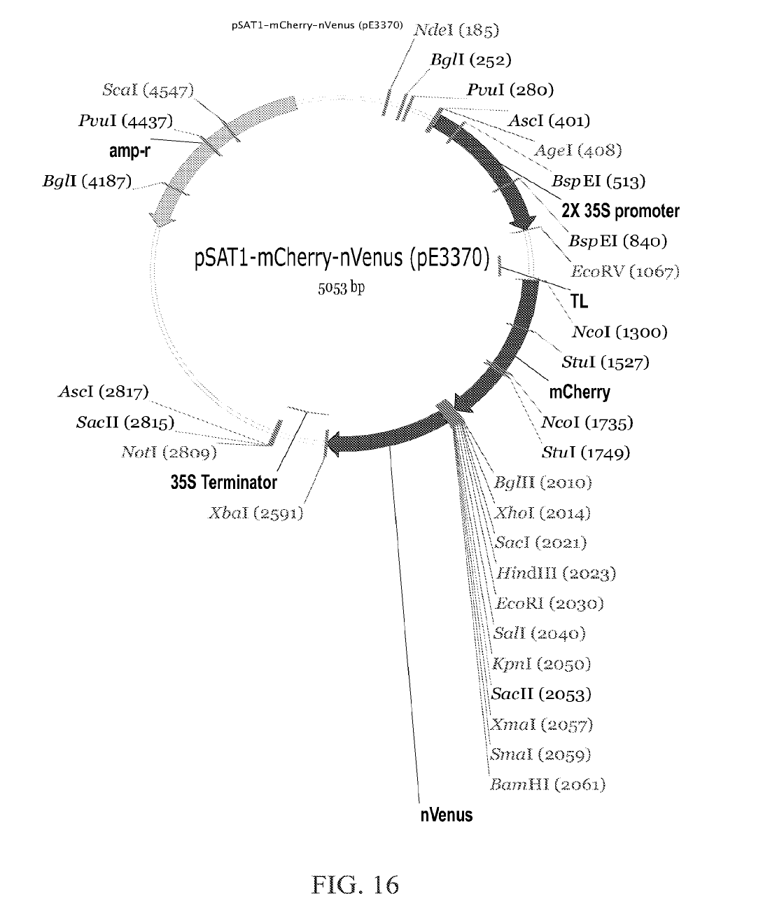

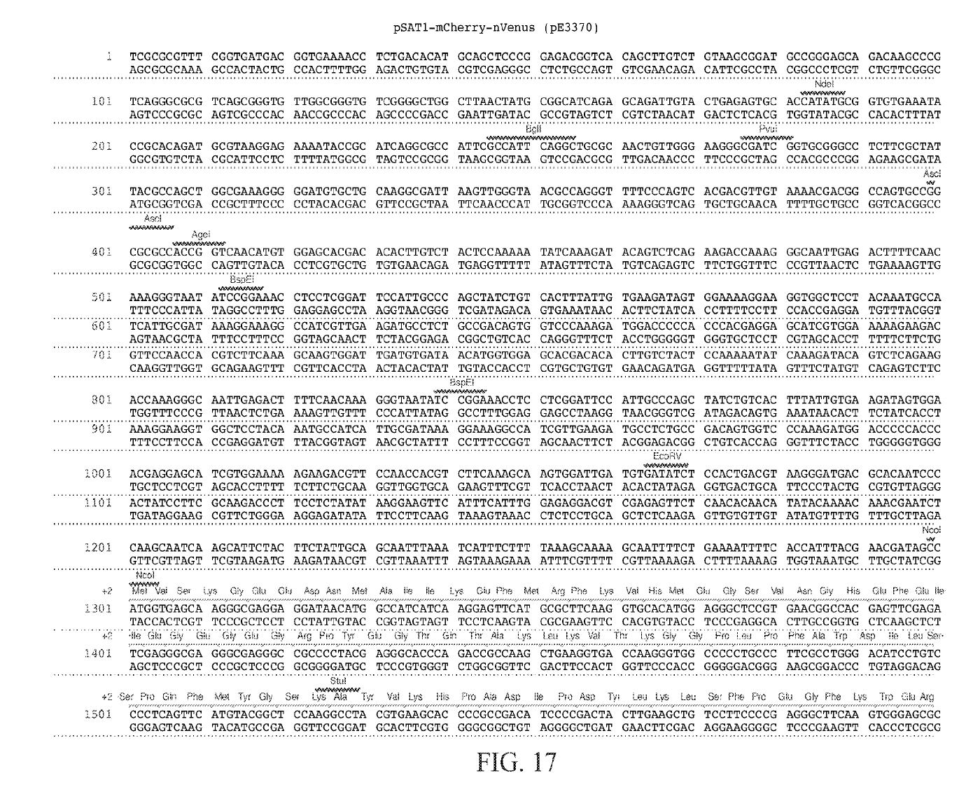

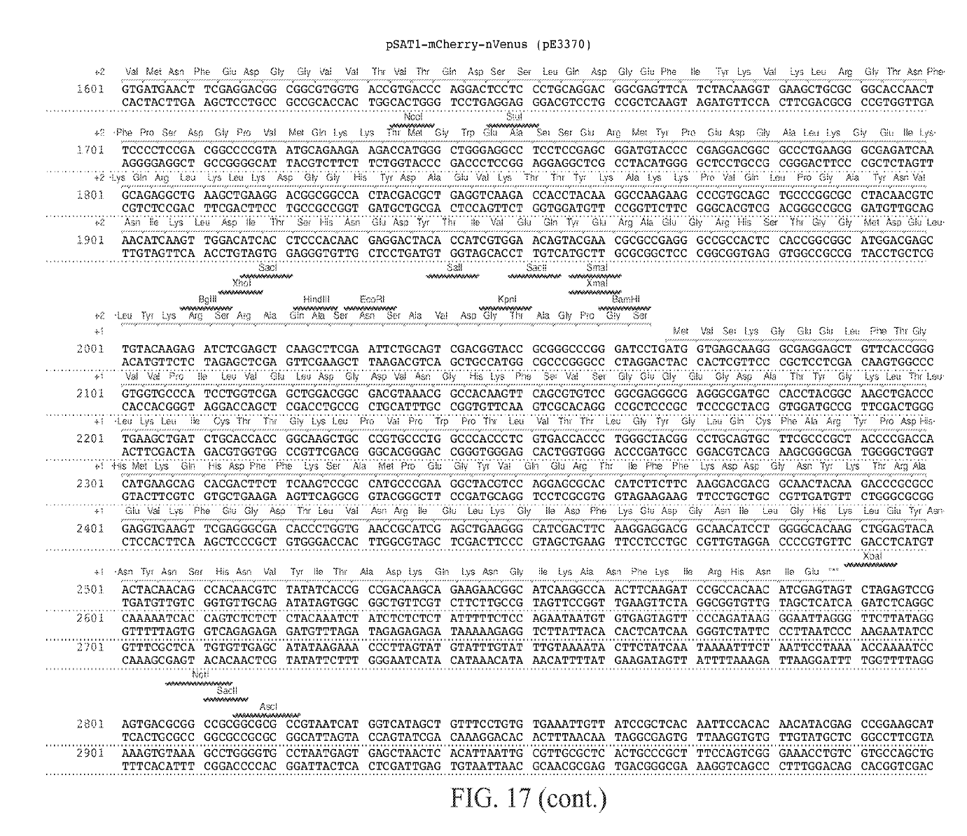

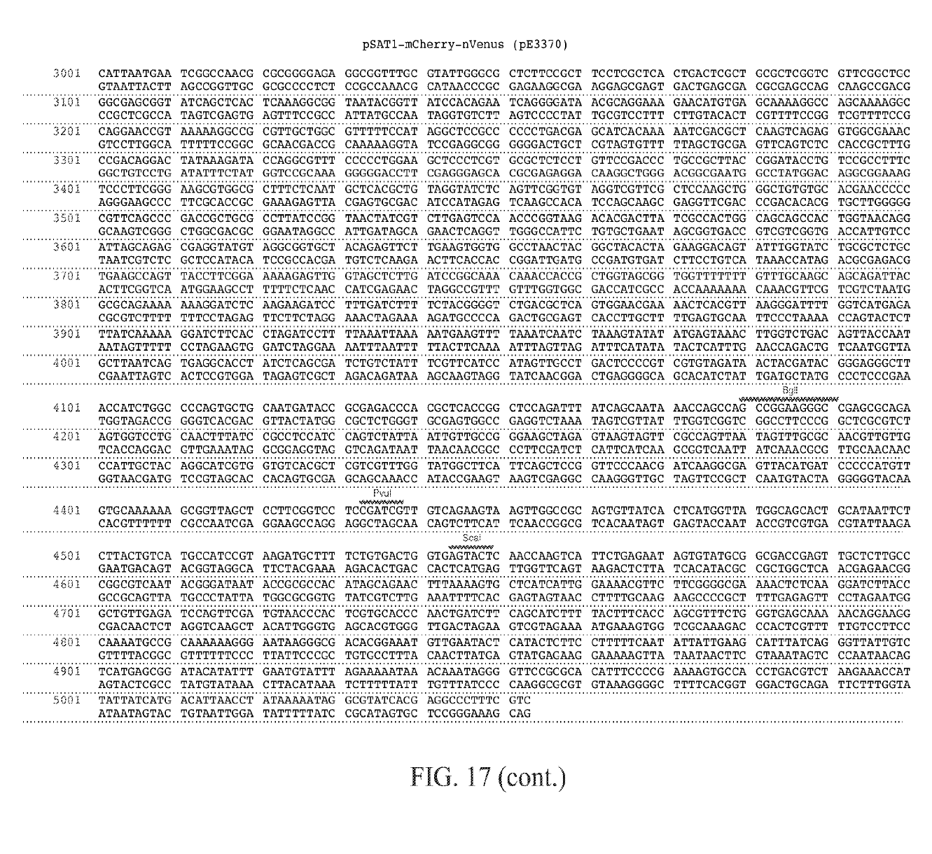

FIG. 15 shows Gateway® attL-flanked oligomers encoding random 20 amino acid aptamers will be inserted as part of the polyprotein.

### EXAMPLES

Examples are provided for illustrative purposes and are not intended to limit the scope of the disclosure.

### Example 1

### BiFC Assay to Detect Aptamer-Protein Interactions in Plant Cells

BiFC was developed to study protein-protein interactions in plant cells. In this approach, a molecule of yellow spectral variant of GFP (YFP) is separated into two portions, N-terminal (nYFP) and C-terminal (cYFP), neither of which fluoresces when expressed alone. Fluorescence is restored when nYFP and cYFP refold, as they are brought together as fusions with interacting proteins. BiFC allows detection of protein-protein interactions in planta and simultaneously determination of the sub-cellular localization of the interacting proteins. BiFC is the basis for detecting peptide aptamer-protein interactions.

FIG. 1 shows the design for the initial aptamer expression cassette. Aptamer coding sequences are inserted into a multiple cloning site (MCS) to generate a polyprotein containing full-length mCherry (lacking a stop codon) at the N-terminus and the N-terminal portion of Venus (nVenus) at the C-terminus (YFP variants, SEYEP-F46L, “Venus”). Expression of the polyprotein is under control of a CaMV double 35S promoter and a TEV translational enhancer. The promoter is flanked by unique AgaI and EcoRV sites such that it can be replaced by other promoters. The entire expression cassette is flanked by rare-cutting Asa sites to facilitate transfer among vectors.

mCherry (Shaner et al., 2004) and Venus (Nagai et al., 2002) arc enhanced, monomeric forms of dsRed and GFP, respectively. As well as “constraining” the aptamer at its N-terminus, mCherry serves as a red fluorescent marker for those cells that have received the aptamer expression cassette. nVenus serves both to “constrain” the aptamer at its C-terminus, and as a non-fluorescent peptide that, when correctly folded with a C-terminal fragment of another GFP derivative, will fluoresce yellow in BiFC. Amino acids 1-174 of nVenus are “paired” with the C-terminal portion of CFP (cCFP, amino acids 155-239). Complementation by this combination of fragments results in the strongest fluorescence signals.

As proof of concept, Agrobacterium VirE2 protein was chosen as the target for aptamer interaction and mutagenesis because: 1) VirE2 is important for defining transformation functions; 2) VirE2 is known to interact with both plant and Agrobacterium proteins in vivo, including itself, VirE1, VirD4, VIP1, VIP2, and various important a proteins. In addition, VirE2 interacts with T-strands; 3) Some of the interacting domains of VirE2 have been mapped in yeast. FIG. 5 shows a map of VirE2 domains important for self-interaction and for interaction with VirE1.

FIG. 6 shows regions of VirE2 from which 20-mer aptamers were made. Aptamer 1 covers non-conserved sequences among VirE2 proteins from different bacterial strains, and therefore may represent a region unimportant for VirE2 function. Aptamers 2-8 represent regions important for VirE2 homopolymerization. Aptamers 6-8 overlap regions important for VirE1-VirE2 interaction. Domain 9 contains the T4SS secretion signal, not likely crucial for VirE2 function in planta. The mid-80 aptamer domain covers one NLS and sequences important for VirE2-VirE2 interactions.

The aptamer expression cassette was co-electroporated (either lacking or containing the various aptamers; see FIG. 1) with a construction expressing VirE2-cCFP into tobacco BY-2 protoplasts and red and yellow fluorescence was visualized 24 hr later. Red (mCherry) fluorescence indicates expression of the aptamer cassette. Note that “free” aptamer (i.e., not interacting with VirE2) localizes both to the cytoplasm and to the nucleus (FIG. 7, “empty vector”). Each aptamer was able to interact with VirE2, resulting in yellow fluorescence. Note that for aptamers 4 and 9 this interaction occurred both in the nucleus and cytoplasm whereas other aptamers interacted mostly with the cytoplasmic VirE2. Taken together, the results indicate that: 1) Aptamer-target interactions can easily be detected and the sub-cellular localization of the interacting polypeptides determined by BiFC in living plant cells; 2) The detected interactions are specific because the mCherry-nVenus “empty vector” polyprotein—which lacks an aptamer sequence and, thus, is not expected to interact with VirE2—produces no non-specific signal. These data support the feasibility of the methods and composition disclosed herein.

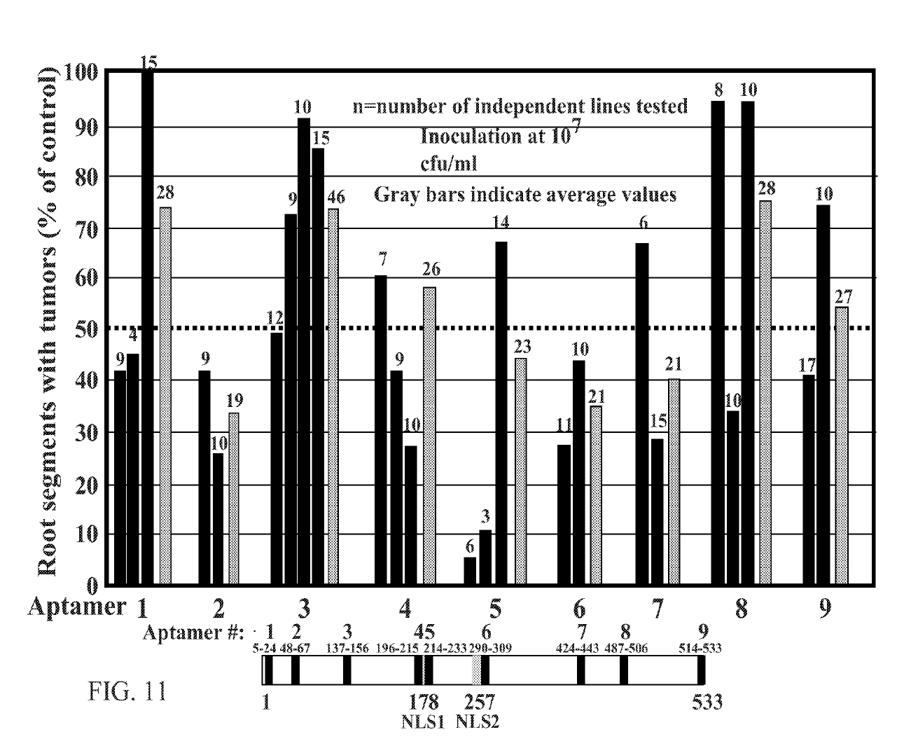

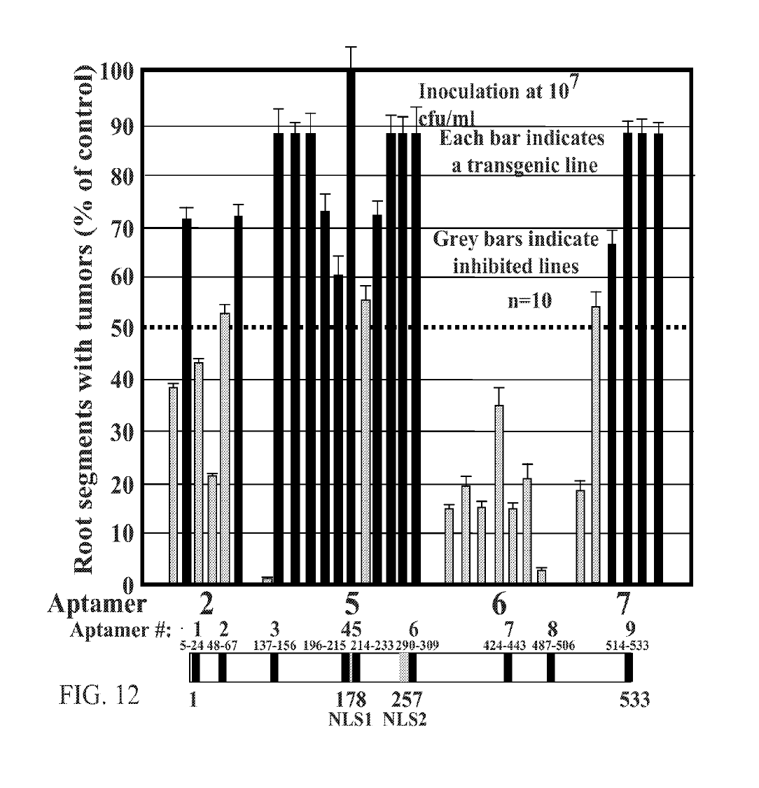

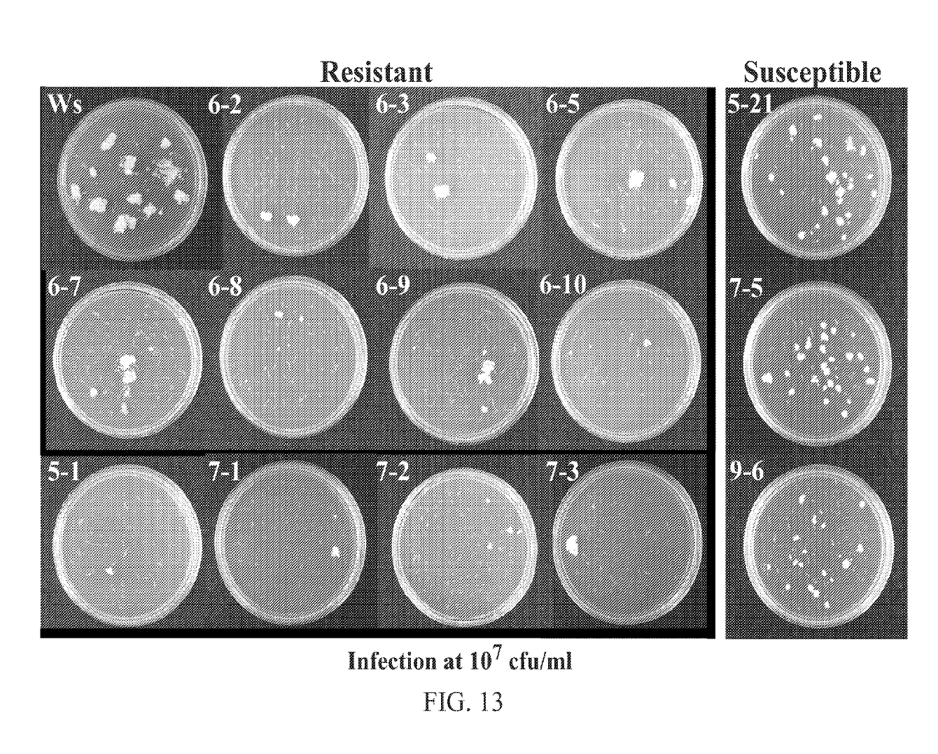

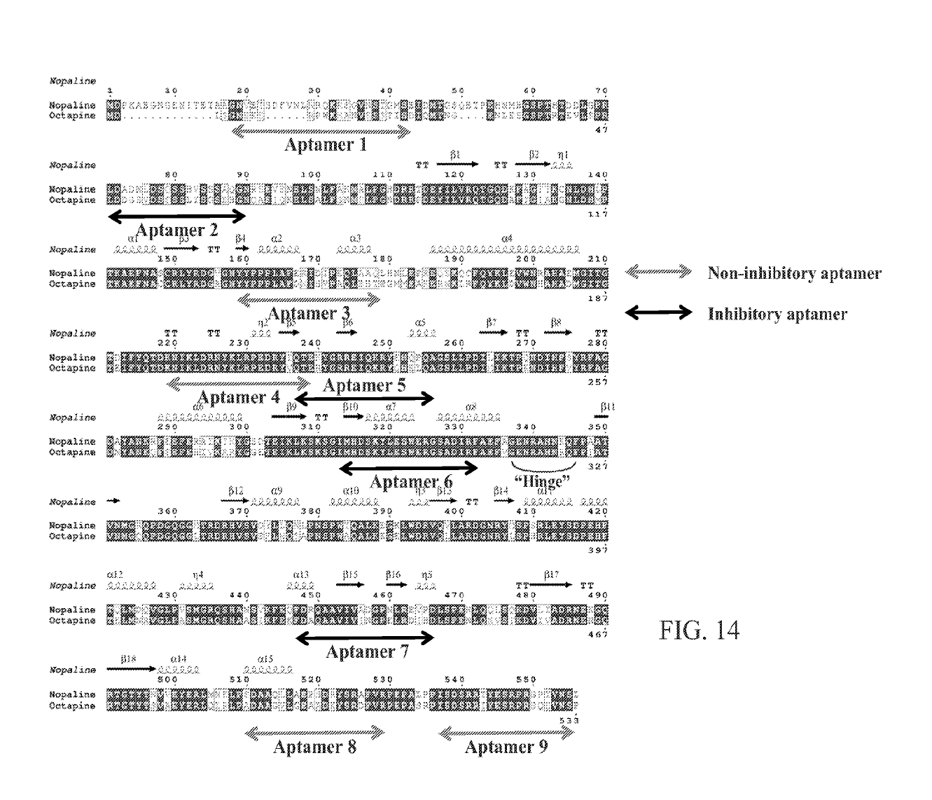

Transgenic Arabidopsis expressing each aptamer polyprotein were generated, and hundreds of plants were assayed for susceptibility to Agrobacterium. The results of these assays (FIG. 11-13) indicate that aptamers 1, 5, 6, and 7 reproducibly inhibited transformation.

### Materials and Methods

Gateway® Recombination Cloning Technology, (Invitrogen by Life Technologies) was used. The typical cloning workflow involves many steps, particularly traditional restriction enzyme cloning. Gateway® recombination cloning uses a one hour, 99%-efficient, reversible recombination reaction, without using restriction enzymes, ligase, subcloning steps, or screening of countless colonies and makes expression-ready clones. Gateway® technology facilitates cloning of genes, into and back out of, multiple vectors via site-specific recombination.

### PUBLICATIONS

These publications are incorporated by reference to the extent they relate materials and methods disclosed herein.
Dym et al. (2008) Crystal structure of the Agrobacterium virulence complex VirE1-VirE2 reveals a flexible protein that can accommodate different partners. Proc. Natl. Acad. Sci. USA 105: 11170-11175.
Hu et al., 2002. Mol. Cell 9:789-798 Visualization of protein-protein interactions in living cells.
Nagai, T., Ibata, K., Park, E. S., Kubota, M., Mikoshiba, K., and Miyawaki, A. (2002) A variant of yellow fluorescent protein with fast and efficient maturation for cell-biological applications. Nature Biotechnol. 20, 87-90.
Shaner, N. C., Campbell, R. E., Steinbach, P. A., Giepmans, B. N., Palmer, A. E., and Tsien, R. Y. (2004) Improved monomeric red, orange and yellow fluorescent proteins derived from Discosoma sp. red fluorescent protein. Nat. Biotechnol. 22, 1567-1572.

1. A method of using dual fluorescent signals to identify a peptide aptamer that specifically interacts with a target protein in a cell, comprising:
a. introducing into said cell at least one said peptide aptamer in an expression cassette, wherein said peptide aptamer is translationally linked to one fluorescent reporting protein at one end and a first partial auto-fluorescent protein fragment selected from a pair of BiFC (Bimolecular Fluorescence Complementation) at the other end;
b. introducing into said cell at least one said target protein, wherein said target protein is translationally linked to a second partial auto-fluorescent protein fragment selected from the pair of BiFC that is complementary to said first partial auto-fluorescent protein fragment;
c. observing a first fluorescent signal produced by said reporting protein to identify said peptide aptamer's expression and location in said cell; and
d. observing a second fluorescent signal produced by said pair of BiFC to confirm said aptamer specifically interacts with said target protein in said cell.

2. The method according to claim 1, wherein the dual fluorescent signals identify the peptide aptamer that disrupts said target protein's function.

3. The method according to claim 1, wherein said expression cassette is inducible or constitutive.

4. The method according to claim 2, wherein said cell is a plant cell.

5. The method according to claim 2, wherein said cell is an animal cell.

6. The method according to claim 5, wherein said cell is a cancer cell.

7. The method according to claim 1, wherein said fluorescent reporting protein is an enhanced form of dsRed.

8. The method according to claim 1, wherein said pair of BiFC are two parts of a yellow spectral variant of GFP consisting of N-terminal (nYFP) and C-terminal (cYFP), or two parts of a YFP consisting of nVenus and cVenus.

9. The method according to claim 4, wherein said target protein is Agrobacterium virulence effector protein designated as VirE2.

10. A method to create a cell having a target protein function null phenotype, comprising introducing said peptide aptamer identified according to claim 2 to said cell.

11. The method according to claim 10, wherein the cell is a plant cell with VirE2 function null and said plant cell is resistant to Agrobacterium mediated plant transformation.

12. The method according to claim 10, wherein the cell is a cancer cell with a cancer mobility protein function null and said cancer cell is restrained from metastasis.


SEQUENCE LISTING (5 sequences)

SEQ ID NO: 1 - protein, 556 aa, Agrobacterium tumefaciens
MDPKAEGNGENITETAAGNVETSDFVNLKRQKREGVNSTGMSEIDMTGSQETPEHNMHGSPTHTDDLGPRLDADMLDSQSSHVSSSAQGNRSEVENELSNLFAKMALPGHDRRTDEYILVRQTGQDKFAGTTKCNLDHLPTKAEFNASCRLYRDGVGNYYPPPLAFERIDIPEQLAAQLHNLEPREQSKQCFQYKLEVWNRAHAEMGITGTDIFYQTDKNIKLDRNYKLRPEDRYIQTEKYGRREIQKRYEHQFQAGSLLPDILIKTPQNDIHFSYRFAGDAYANKRFEEFERAIKTKYGSDTEIKLKSKSGIMHDSKYLESWERGSADIRFAEFAGENRAHNKQFPAATVNMGRQPDGQGGMTRDRHVSVDYLLQNLPNSPWTQALKEGKLWDRVQVLARDGNRYMSPSRLEYSDPEHFTQLMDQVGLPVSMGRQSHANSVKFEQFDRQAAVIVADGPNLREVPDLSPEKLQQLSQKDVLIADRNEKGQRTGTYTNVVEYERLMMKLPSDAAQLLAEPSDRYSRAFVRPEPALPPISDSRRTYESRPRGPTVNSL

SEQ ID NO: 2 - protein, 533 aa, Agrobacterium tumefaciens
MDLSGNEKSRPWKKANVSSSTISDIQMTNGENLESGSPTRTEVLSPRLDDGSVDSSSSLYSGSEHGNQAEIQKELSALFSNMSLPGNDRRPDEYILVRQTGQDAFTGIAKGNLDHMPTKAEFNACCRLYRDGAGNYYPPPLAFDKISVPAQLEETWGMMEAKERNKLRFQYKLDVWNHAHADMGITGTEIFYQTDKNIKLDRNYKLRPEDRYVQTERYGRREIQKRYQHELQAGSLLPDIMIKTPKNDIHFVYRFAGDNYANKQFSEFEHTVKRRYGGETEIKLKSKSGIMHDSKYLESWERGSADIRFAEFVGENRAHNRQFPTATVNMGQQPDGQGGLTRDRHVSVEFLMQSAPNSPWAQALKKGELWDRVQLLARDGNRYLSPHRLEYSDPEHFTELMNRVGLPASMGRQSHAASIKFEKFDAQAAVIVINGPELRDIHDLSPENLQNVSTKDVIVADRNENGQRTGTYTSVAEYERLQLRLPADAAGVLGEAADKYSRDFVRPEPASRPISDSRRIYESRPRSQSVNSF

SEQ ID NO: 3 - DNA, 5053 bp, Artificial Sequence
  Description of Artificial Sequence Synthetic polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccggcgcgccaccggtcaacatgtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacgtcgagagttctcaacacaacatatacaaaacaaacgaatctcaagcaatcaagcattctacttctattgcagcaatttaaatcatttcttttaaagcaaaagcaattttctgaaaattttcaccatttacgaacgatagccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagagatctcgagctcaagcttcgaattctgcagtcgacggtaccgcgggcccgggatcctgatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgagtagtctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacgcggccgcggcgcgccgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 4 - protein, 255 aa, Artificial Sequence
  Description of Artificial Sequence Synthetic polypeptide
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKRSRAQASNSAVDGTAGPGS

SEQ ID NO: 5 - protein, 173 aa, Artificial Sequence
  Description of Artificial Sequence Synthetic polypeptide
MVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIE